(19) United States
(12) Patent Application Publication
(10) Pub. No.: US 2003/0100891 A1
(43) Pub. Date: May 29, 2003

(54) Surgical console macro recorder

(76) Inventor: Kirk Todd, Yorba Linda, CA (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/003,007

(22) Filed: Oct. 23, 2001

(51) Int. Cl. A61B 17/00

(52) U.S. Cl. 606/1

(57) ABSTRACT

A surgical console containing a macro recording program. Once initiated, the program records the various operating parameters being used by the surgeon during a surgical procedure. This macro can then be used by the surgeon to set the operating parameters of the surgical console in future, similar surgeries.

### BACKGROUND OF THE INVENTION

This invention relates generally to the field of surgical consoles and, more particularly, to microsurgical consoles.

During modem surgery, particularly ophthalmic surgery, the surgeon uses a variety of pneumatic and electronically driven microsurgical handpieces. The handpieces are operated by a microprocessor-driven surgical console that receives inputs from the surgeon or an assistant by a variety of peripheral devices, such as footpedal controllers, infrared remote control devices and menu-driven touchscreens. One such microsurgical console is described in U.S. Pat. No. 5,455,766 (Scheller, et al.), the entire contents of which is incorporated herein by reference. The device disclosed in this patent allows the operating parameters of the various surgical devices to be stored in the memory of the console and/or downloaded onto magnetic media and transferred to other surgical consoles. Other surgical consoles, such as the SERIES TWENTY THOUSAND® LEGACY® surgical system and the ACCURUS® surgical system, both available from Alcon Laboratories, Inc., allow for surgeons to manually input surgical operating parameters and store those “customized” parameters in the console memory for future use. These prior art devices, however, all require that the operating parameters be inputted manually using a keypad or touchscreen or downloaded from another console that has had the parameters inputted manually.

Accordingly, a need continues to exist for a surgical console that allows for the programming of the surgical operating parameters without the use of a keypad, a touchscreen or external media.

### BRIEF SUMMARY OF THE INVENTION

The present invention improves upon the prior art surgical consoles by providing a surgical console containing a macro recording program. Once initiated, the program records the various operating parameters being used by the surgeon during a surgical procedure. This macro can then be used by the surgeon to set the operating parameters of the surgical console in future, similar surgeries.

Accordingly, one objective of the present invention is to provide a surgical console having a macro recorder.

Another objective of the present invention is to provide a surgical console capable of saving operating parameters into its memory without the use of a keypad, a touchscreen or external media.

These and other advantages and objectives of the present invention will become apparent from the detailed description and claims that follow.

### BRIEF DESCRIPTION OF THE DRAWINGS

FIG. 1 is a perspective view of one surgical console that may be used with the present invention.

FIG. 2 is a flow chart indicating the steps followed by a doctor in recording a surgical procedure macro as contemplated by the present invention.

FIG. 3 is a flow chart indicating the steps followed by a doctor in playing a recorded surgical procedure macro as contemplated by the present invention.

FIGS. 4a-4c are block diagrams depicting the steps employed by the macro recorder of the present invention.

### DETAILED DESCRIPTION OF THE INVENTION

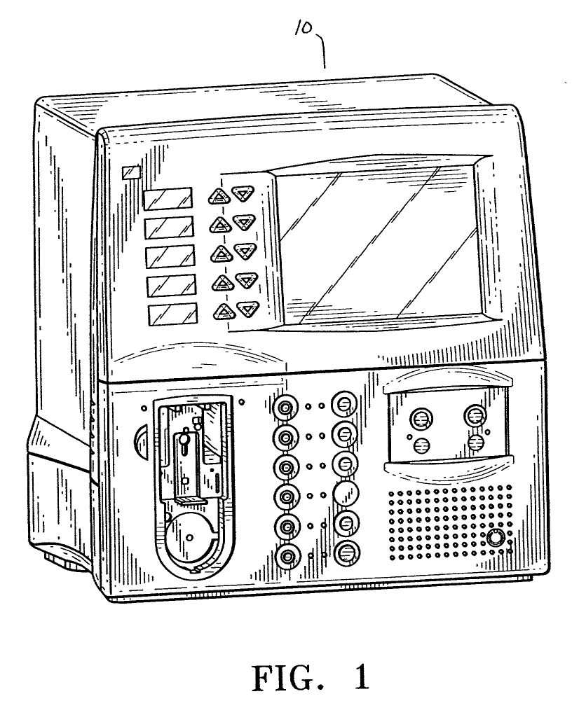

The macro recorder of the present invention may be used with any suitable surgical console as the SERIES TWENTY THOUSAND® LEGACY® surgical system console or the ACCURUS® surgical system console, as seen in FIG. 1, both commercially available from Alcon Laboratories, Inc., Fort Worth, Tex., that contain programmable memories for storing operating parameters.

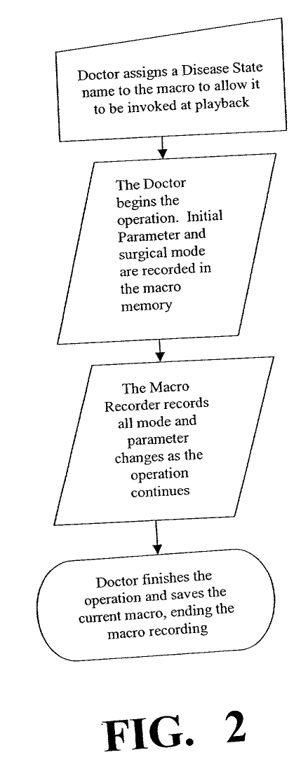

As seen in FIG. 2, the macro recorder of the present invention is initiated by the surgeon assigning a name, such as a Disease State to the macro to be recorded, and the recorder is initialized. The surgeon then begins the surgical procedure, and the initial operating parameters and modes are saved into console 10 memory by the recorder. As the surgeon continues through the surgical procedure, changes in the operating modes and peripheral device operating parameters are saved by the recorder into the console memory while the procedure is being conducted. When the surgical procedure is completed, the macro is saved in the memory of console 10 and the recorder turned off.

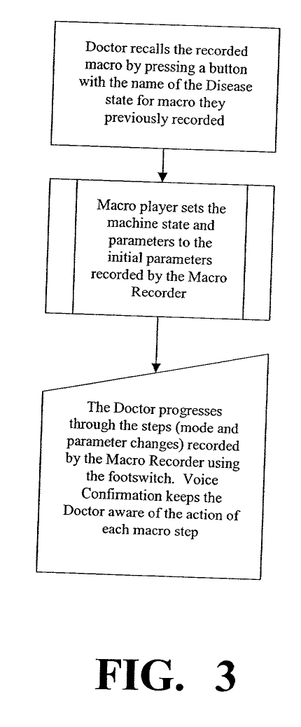

As seen in FIG. 3, in use, when the surgeon wants to use a previously recorded macro, the surgeon recalls the macro from the console memory and initializes the macro. The macro then automatically adjust the settings and operating parameters of the console are required by the surgical procedure. Scrolling through the various steps in the macro can be accomplished by use of the console footswitch, remote control or console touchscreen. The commands may be confirmed via voice confirmation by the console.

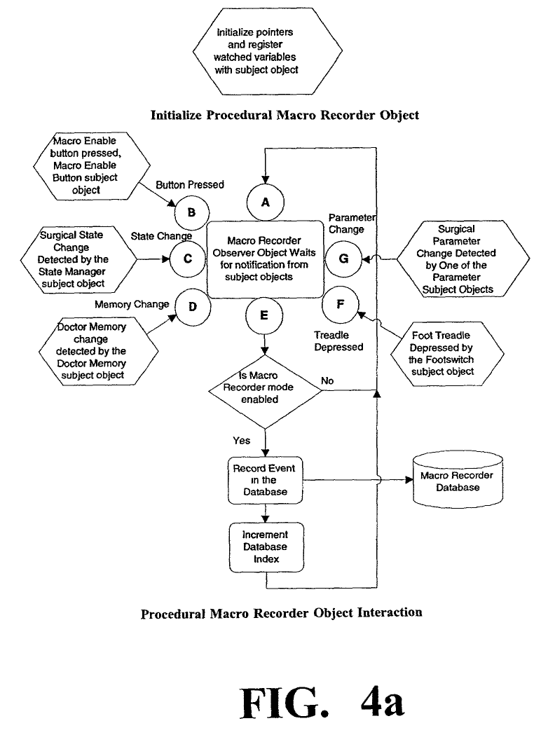

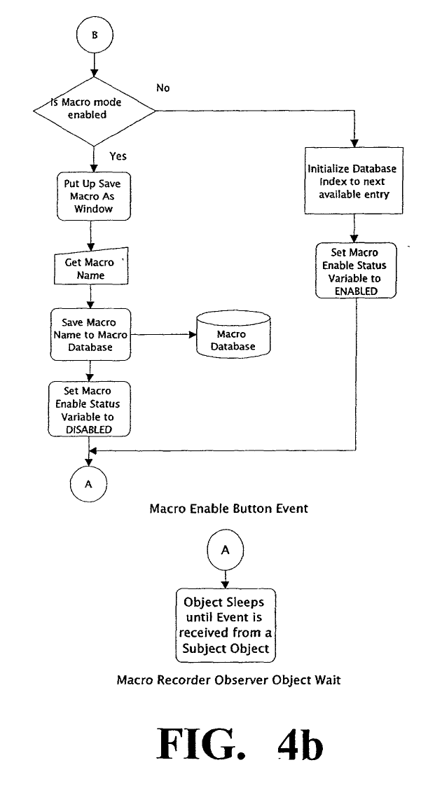

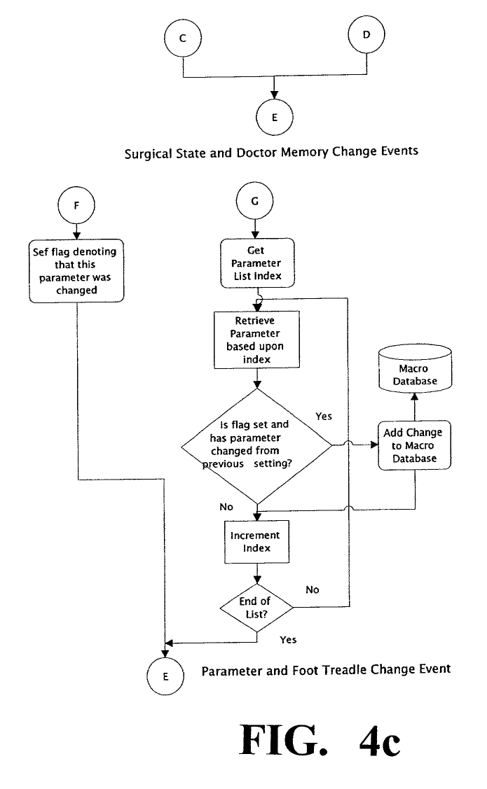

The logic employed by the macro recorder of the present invention is illustrated in FIGS. 4a-4c.

The macro recorder of the present invention may be implemented using software commands well-known in the art and does not require any hardware modification to the commercially available surgical consoles. The logic employed by the macro recorder of the present invention is illustrated in FIGS. 4a-4c.

This description is given for purposes of illustration and explanation. It will be apparent to those skilled in the relevant art that modifications may be made to the invention as herein described without departing from its scope or spirit.

I claim:

1. A method of operating a surgical console, comprising the steps of:
a) initialized a macro recorder, the macro recorder being resident in the surgical console;
b) conducting a first surgical procedure using the surgical console;
c) recording surgical console operating parameters and surgical modes used during the first surgical procedure on the macro recorder as the first surgical procedure is being performed; and
d) saving the surgical console operating parameters and surgical modes used during the surgical procedure as a macro.

2. The method of claim 1 further comprising the step of recalling the macro and conducting a second surgical procedure using the surgical console operating parameters and surgical modes as saved in the macro.
